(19) United States
(12) Patent Application Publication
(10) Pub. No.: US 2010/0185223 A1
(43) Pub. Date: Jul. 22, 2010

(54) SWITCHING STICK WITH HOODED BLADE

(76) Inventor: John S. Rogerson, Madison, WI (US)

Correspondence Address: Intellectual Property Dept.;Dewitt Ross & Stevens SC, 2 East Mifflin Street, Suite 600, Madison, WI, 53703-2865, US

(21) Appl. No.: 12/619,183

(22) Filed: Nov. 16, 2009

Related U.S. Application Data
(60) Provisional application No. 61/114,607, filed on Nov. 14, 2008.

(51) Int. Cl. A61B 17/32

(52) U.S. Cl. 606/170

(57) ABSTRACT

A surgical switching stick is described. The switching stick includes a handle member, a shoulder continuous with the distal end of the handle member and progressively decreasing in girth toward a distal end of the switching stick, a curved tip located at a distal end of the shoulder and defining a space between an inner curved portion of the curved tip and the shoulder, and a blade disposed in the space. Methods for using the switching stick for controllably widening an incision are also described.

### CROSS REFERENCE TO RELATED APPLICATIONS

This application claims priority under 35 USC §119(e) to U.S. Provisional Patent Application 61/114,607 filed Nov. 14, 2008, the entirety of which is incorporated herein by reference.

### FIELD OF THE INVENTION

The invention relates to a surgical switching stick for creating and dilating an incision into a body cavity such as a joint capsule. The primary utility of the switching stick is to facilitate entry of surgical instruments into a confined body location (e.g., a joint).

### BACKGROUND

Minimally invasive surgical techniques have made impressive advances in recent decades. Tools such as arthroscopes, laparoscopes, and the like and surgical techniques employing these tools have changed what were previously major surgeries (e.g., arthrotomy) that required hospitalization and extensive recuperation time into out-patient procedures that have the patient back home the same day. Minimally invasive surgery results in less trauma to, and hence less healing time for, the patient as compared to corresponding open procedures.

Minimally invasive surgical procedures are conducted through a small, dilated incision that provides access to a body cavity or area requiring attention. While this approach is beneficial for the patient, it is often difficult and tedious for the surgeon to accomplish the goals of the surgery via such a small access aperture. It is often the case that an initial incision must be dilated further in mid-procedure to provide the surgeon the required access to the surgical site. This is especially the case in procedures involving encapsulated joints, such as knees, hips, and shoulders. To access these joints, the capsule surrounding the joint must be pierced. If the access aperture through the capsule is too small, or slightly misaligned, it must be widened to allow the surgical instruments to be properly inserted into the joint.

Typically, an initial access point is formed by making a first, very small incision. The initial incision is then widened using a conventional surgical dilator, such as that shown in U.S. Pat. No. 5,342,384. A retractor may then be used to keep the dilated incision open while the surgery proceeds. However, in the process of dilating the incision, the opening through the membrane or other tissue being dilated will sometimes expand too far, too fast, or in an otherwise unwanted and/or uncontrolled fashion. Therefore, there remains a need for a surgical dilator that allows for the controlled dilation of a surgical incision.

### SUMMARY OF THE INVENTION

To overcome the shortcomings of the devices and methods described above, the present invention is directed to a switching stick that includes a handle member having a proximal and a distal end, a shoulder having a proximal and a distal end wherein the proximal end of the shoulder is continuous with the distal end of the handle member and the shoulder progressively decreases in girth toward its distal end, a curved tip located at the distal end of the shoulder and defining a space between an inner curved portion of the curved tip and the shoulder, and a blade disposed in the space between the inner curved portion of the curved tip and the shoulder.

In one version of the invention, the handle member is elongated between its proximal and distal ends, is cylindrical in shape, and/or further includes a textured surface to facilitate gripping of the switching stick.

In another version of the invention, the switching stick defines a longitudinal axis, a first horizontal axis, and a second horizontal axis, each axis being orthogonal to the other axes, wherein the curved tip extends from the shoulder along the longitudinal axis and curves with respect to only the first horizontal axis.

In yet another version of the invention, the switching stick defines a longitudinal axis, a first horizontal axis, and a second horizontal axis, each axis being orthogonal to the other axes, wherein a most distal end of the curved tip is substantially perpendicular to the longitudinal axis.

In some versions of the invention, the curved tip defines a continuous outer curved portion.

In another version of the invention, the curved tip, shoulder, and handle member together include at least one continuous surface.

In yet other versions of the invention, the blade fills the entire space between the inner curved portion of the curved tip and the shoulder, and/or the blade includes at least one exposed edge. In a preferred version, the blade has only one exposed edge. The exposed edge of the blade may be either a straight edge or be curved.

In one version of the invention, the curved edge includes a slot for inserting the blade in the space between the inner curved portion of the curved tip and the shoulder.

The blade may be dimensioned and configured to be either reversibly or permanently attached to the switching stick.

The different components of the switching stick, including the handle member, shoulder, and/or the curved tip may be fabricated from a variety of materials, including metal and plastic.

The invention also includes methods of controllably widening an incision. One method includes inserting a switching stick as described herein in an incision and contacting an exposed edge of the blade against a boundary of the incision to widen the incision.

Other methods further include, or alternatively include, a step of contacting only the curved tip, shoulder, and/or handle member, rather than the blade, against surrounding tissue of the incision to widen the incision.

The invention provided herein permits widening of an incision in a controlled fashion.

The objects and advantages of the invention will appear more fully from the following detailed description of the preferred embodiment of the invention made in conjunction with the accompanying drawings.

### BRIEF DESCRIPTION OF THE DRAWINGS

FIG. 1 is a perspective rendering of a preferred version of the presently claimed surgical dilator or switching stick.

FIG. 2 is a top plan view of the preferred version of the invention depicted in FIG. 1.

FIG. 3 is front elevation view of the preferred version of the invention depicted in FIG. 1.

### DETAILED DESCRIPTION OF THE INVENTION

The invention is directed to a medical dilator or switching stick for creating and dilating an incision in muscles, membranes, or other tissues. As used herein, the terms “dilator” and “switching stick” are used synonymously.

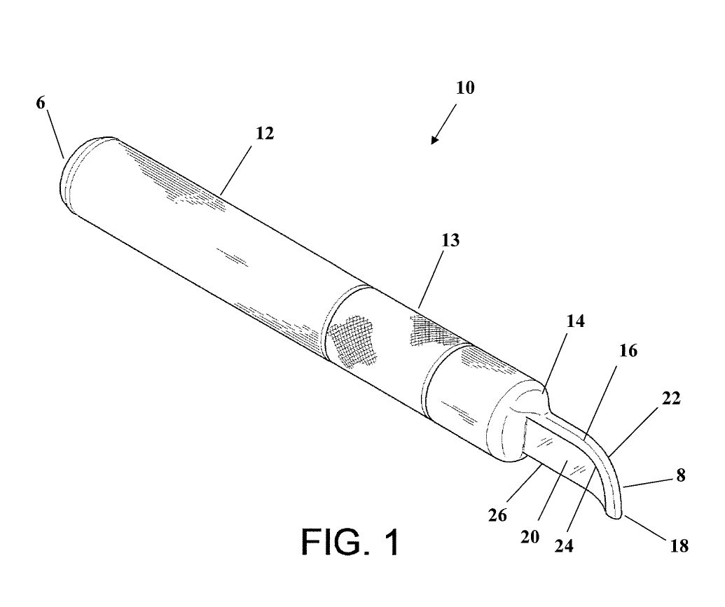

Referring now to the figures, where the same reference numerals are used throughout to designate the various features of the invention, FIGS. 1-3 depict an exemplary switching stick 10 according to the present invention. The exemplary switching stick 10 has proximal 6 and distal 8 ends and includes, most proximally, a handle member 12, a shoulder 14 disposed on the distal end of the handle member 12, a curved tip 16 extending from the shoulder 14, and a blade 20 attached to the curved tip 16.

The handle member 12 is dimensioned and configured to be held by a user and to be inserted within incisions. For applications involving small incisions, the handle member 12 is structured to be held with just the fingers of the hand. For applications involving large incisions, the handle member 12 may be structured to be grasped with the user's entire hand. As used herein, “structured” means both dimensioned and configured. In a preferred version, the handle member 12 has a rod-like shape with an extended longitudinal axis relative to its horizontal axes. The handle member 12 may be straight or curved along its longitudinal axis. The cross section of the handle member 12 may comprise any two-dimensional shape, including circles, ovals, polygons, etc. The shapes comprising the cross section of the handle member 12 may be symmetrical or non-symmetrical. Several acceptable rod-like shapes include cylindrical and cuboidal shapes. The exemplary version of the invention shown in the figures is cylindrical. The handle member 12 may optionally include a textured surface 13 to facilitate gripping of the switching stick 10.

The shoulder 14 is disposed on the distal end of the handle member 12 and is continuous with it. As used herein, “continuous” refers to surfaces lacking substantially defined edges, corners, or sharp topographical changes, save for gradual surface curvature. Thus, the portion of the shoulder 14 meeting the handle member 12 has a cross-sectional size and shape that matches that of the distal end of the handle member 12. The shoulder 14 progressively decreases in girth toward the distal end 8 of the switching stick 10. The progressive change in girth functions as a dilator when the distal end of the switching stick 10 is inserted into a surgical incision that is smaller than the diameter of the handle member 12. The progressive change in girth serves, in part, to dilate incisions in tissue when the distal end of the switching stick 10 is inserted therein. The progressive change in girth may be abrupt or gradual depending on the type of incision and tissue with which it is used.

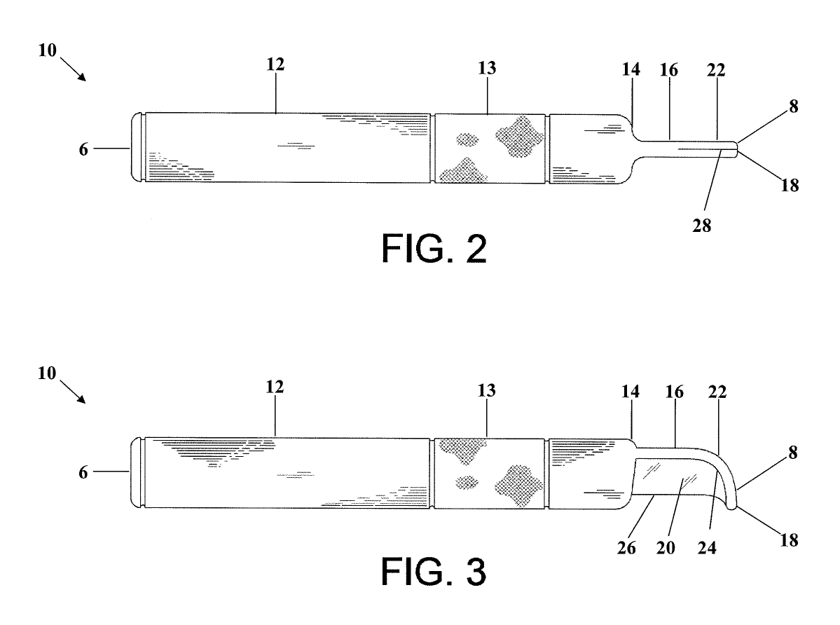

The curved tip 16 is continuous with, and extends longitudinally from, the shoulder 14. The curved tip 16 is preferably curved with respect to only one of two orthogonal horizontal axes of the switching stick 10. In other words, the curved tip 16 curves within a plane defined by the longitudinal axis and a first of two orthogonal horizontal axes, as depicted in FIG. 3, but extends in a straight line within a plane defined by the longitudinal axis and a second of the two orthogonal horizontal axes, as shown in FIG. 2. The curvature of the curved tip 16 is preferably sufficient to orient the most distal end 18 of the curved tip 16 substantially perpendicular to the longitudinal axis of the switching stick 10. The curvature of the curved tip 16 defines an inner curved portion 24 and an outer curved portion 22. The outer curved portion 22 preferably defines a continuous, smooth surface lacking edges so that it can be inserted into incisions without tearing or slicing surrounding tissue.

The curved tip 16, shoulder 14, and handle member 12 together preferably comprise at least one continuous surface. In the exemplary version, the continuous surface begins at the most distal end 18 of the curved tip 16 and continues along the outer curved portion 22 of the curved tip 16, the area of increasing girth of the shoulder 14, and at least a portion of the handle member 12. The continuous surface allows the switching stick 10 to be inserted into incisions without tearing or slicing surrounding tissue.

The blade 20 is disposed in the space defined between the inner curved portion 24 of the curved tip 16 and the distal end of the shoulder 14. The blade 20 preferably takes up the entire space therein. The curved tip 16 is structured both to hold the blade 20 in place and to shield the upper surface of the blade from contacting the boundaries of an incision. In one version of such a structure, the curved tip 16 extends along a first edge or edges of the blade 20 from the shoulder 14 to the most distal end 18 of the curved tip 16 such that only a single edge 26 of the blade 20 is exposed. In this fashion, the distal end 8 of the switching stick can be inserted into an incision without the blade 20 making contact with the boundaries of the incision. The exposed edge 26 may be a straight edge, or may have a curvature. The exposed edge 26 preferably has a curvature matching or at least resembling the curvature of the curved tip 16 to minimize unintended, incidental contact with tissue.

The blade may be fabricated from any suitably stiff material, stainless steel being preferred. The blade 20 is fixed to the switching stick 10 by any attachment mechanism now known or developed in the future. The blade 20 may be either permanently or removably attached to the switching stick 10. The blade 20 may be permanently attached using adhesive, solder, or via welding. The blade 20 may be removably attached by means of friction or a transverse pin (not shown) that may pass through corresponding apertures (not shown) in both the curved tip 16 and the top of the blade 20. If removably attached, the blade can be replaced with fresh, sharp blades after use or with blades having different dimensions or configurations for specific applications. The curved tip 16 preferably includes an axial slot 28 to facilitate insertion of the blade 20 within the inner curved portion 24 of the curved tip 16. The axial slot 28 may be included whether the blade 20 is permanently or removably attached.

The unique characteristics of the switching stick 10 described herein allow it to be used both as a conventional dilator and as a device to widen an incision in an extremely controlled fashion. In its preferred use, an initial incision is created with a conventional scalpel or trocar. The device 10 is then inserted into the initial incision, using the outer curved portion 22 of the curved tip 16 and the shoulder 14 to widen the incision gently. If the initial incision proves too confining, the blade 20 can be used to make the initial incision wider by bringing the exposed edge 26 to bear against the boundaries of the incision. This allows the incision to be widened in a controlled fashion using the blade 20. This method is contrasted to the general practice of inserting a dilator of greater diameter into the initial incision.

Switching sticks of the type described herein may be fabricated in sets of progressively increasing girth and can be used sequentially (if desired). As shown in the figures, only a single switching stick is shown, without dimensions. The switching stick can be fabricated in any size based upon its intended surgical use. The switching stick may be fabricated from any suitable material (e.g., steel or other metals, plastic, and the like) and may be designed so as to be a single-use disposable instrument or as an instrument that can be sterilized for repeated use.

The devices and methods of the present invention can comprise, consist of, or consist essentially of the essential elements, limitations, and steps described herein, as well as any additional or optional elements, limitations, or steps described herein or otherwise useful in the art.

As used in this specification and the appended claims, the singular forms “a,” “an,” and “the” include plural referents unless the content clearly dictates otherwise. The term “or” is generally employed in its sense including “and/or” unless the content clearly dictates otherwise.

All publications, patents and patent applications are herein expressly incorporated by reference to the same extent as if each individual publication or patent application was specifically and individually indicated by reference. In case of conflict between the present disclosure and the incorporated patents, publications, and references, the present disclosure should control.

All combinations of method or process steps as used herein can be performed in any order, unless otherwise specified or clearly implied to the contrary by the context in which the referenced combination is made.

The invention is not confined to the particular construction and arrangement of parts herein illustrated and described, but embraces such modified forms thereof as come within the scope of the following claims.

What is claimed is:

1. A switching stick comprising:
a handle member having a proximal and a distal end;
a shoulder having a proximal and a distal end, wherein the proximal end of the shoulder is continuous with the distal end of the handle member and the shoulder progressively decreases in girth toward its distal end;
a curved tip located at the distal end of the shoulder and defining a space between an inner curved portion of the curved tip and the shoulder; and
a blade disposed in the space between the inner curved portion of the curved tip and the shoulder.

2. The switching stick of claim 1 wherein the handle member is elongated between its proximal and distal ends.

3. The switching stick of claim 1 wherein the handle member is cylindrical in shape.

4. The switching stick of claim 1 wherein the handle member further includes a textured surface to facilitate gripping of the switching stick.

5. The switching stick of claim 1 wherein the switching stick defines a longitudinal axis, a first horizontal axis, and a second horizontal axis, each axis being orthogonal to the other axes, wherein the curved tip extends from the shoulder along the longitudinal axis and curves with respect to only the first horizontal axis.

6. The switching stick of claim 1 wherein the switching stick defines a longitudinal axis, a first horizontal axis, and a second horizontal axis, each axis being orthogonal to the other axes, wherein a most distal end of the curved tip is substantially perpendicular to the longitudinal axis.

7. The switching stick of claim 1 wherein the curved tip defines a continuous outer curved portion.

8. The switching stick of claim 1 wherein the curved tip, shoulder, and handle member together include at least one continuous surface.

9. The switching stick of claim 1 wherein the blade fills the entire space between the inner curved portion of the curved tip and the shoulder.

10. The switching stick of claim 1 wherein the blade includes at least one exposed edge.

11. The switching stick of claim 10 wherein the exposed edge is a straight edge.

12. The switching stick of claim 10 wherein the exposed edge is curved.

13. The switching stick of claim 1 wherein the blade has only one exposed edge.

14. The switching stick of claim 1, further comprising a slot in the curved edge for inserting the blade in the space between the inner curved portion of the curved tip and the shoulder.

15. The switching stick of claim 1, wherein the blade is dimensioned and configured to be reversibly attached to the switching stick.

16. The switching stick of claim 1, wherein the blade is dimensioned and configured to be permanently attached to the switching stick.

17. The switching stick of claim 1, wherein the handle member, shoulder, and/or curved tip is fabricated from a metal.

18. The switching stick of claim 1, wherein the handle member, shoulder, and/or curved tip is fabricated from a plastic.

19. A method of controllably widening an incision comprising:
inserting a switching stick as described in claim 1 in an incision; and
contacting an exposed edge of the blade against a boundary of the incision to widen the incision.

20. The method of claim 19 wherein the inserting step includes contacting only the curved tip, shoulder, and/or handle member against surrounding tissue of the incision.
